(19) United States
(12) Patent Application Publication
(10) Pub. No.: US 2009/0240192 A1
(43) Pub. Date: Sep. 24, 2009

(54) INSUFFLATION OF BODY CAVITIES

(76) Inventors: John Sylvester Power, Moycullen (IE); Niall Scott Smith, Alloa (GB); Conor Paul Duffy, Roscahill (IE); Keith Gibbons, Doughiska (IE); Pierre Auguste Diemunsch, Strasbourg (FR)

Correspondence Address: CHOATE, HALL & STEWART LLP, TWO INTERNATIONAL PLACE, BOSTON, MA, 02110, US

(21) Appl. No.: 12/412,740

(22) Filed: Mar. 27, 2009

Related U.S. Application Data
(63) Continuation-in-part of application No. 12/058,255, filed on Mar. 28, 2008.
(60) Provisional application No. 60/907,311, filed on Mar. 28, 2007.
(60) Provisional application No. 61/100,510, filed on Sep. 26, 2008.

(51) Int. Cl. A61M 37/00

(52) U.S. Cl. 604/26

(57) ABSTRACT

Apparatus used in insufflation comprises an insufflator 12 for generating an insufflation gas such as carbon dioxide and an aerosol generator 2 for aerosolising a fluid and entraining the aerosol with the insufflation gas which is delivered during surgery. The aerosol generator 2 comprises a vibratable member 40 having a plurality of apertures extending between a first surface and a second surface. The fluid may comprise a therapeutic or prophylactic agent. A controller 3 is used to control the operation of the aerosol generator 2. The controller 3 controls operation of the aerosol generator 2 responsive to the flow of insufflation gas such as detected by a flow sensor 11. The pulse rate at a set frequency of vibration of the vibratable member 40 is controlled.

### CROSS-REFERENCES TO RELATED APPLICATIONS

The present application is a continuation-in-part of U.S. patent application Ser. No. 12/058,255 filed Mar. 28, 2008 which claims the benefit of U.S. Provisional Application No. 60/907,311 filed Mar. 28, 2007. The present application also claims the benefit of U.S. Provisional Application No. 61/100,510 filed Sep. 26, 2008. The complete contents of all of these are incorporated herein by reference.

### BACKGROUND OF THE INVENTION

Laparoscopic surgery, also called minimally or less invasive surgery (MIS or LIS) or keyhole surgery is a modern surgical technique in which operations in the body are performed through small incisions as compared to the larger incisions needed in traditional surgical procedures. Gas such as carbon dioxide is delivered, via an insufflator, into a body cavity such as the abdomen leading to the formation of a pneumoperitoneum, thereby providing sufficient space for the surgeon to operate. The insufflator maintains the pneumoperitoneum and acts to renew the gas when leaks occur.

Gas such as carbon dioxide that is used for insufflation is both cold and dry and it is not surprising therefore those patients undergoing laparoscopic procedures often suffer a significant drop in core body temperature, which can result in considerable post-surgical pain and significant complications, such as cardiac stress, immunological and clotting problems, for the patient. By using standard thermo physical principles it has been shown that the major cause of patient heat loss is due to evaporation from the body acting to humidify the large volumes of dry insufflated gas at ATPD (Ambient Temperature Pressure Dry) passing into the body which is at BTPS (Body Temperature Pressure Saturated). If such heat loss could be minimised, post-operative pain and the significant side effects suffered by the patient could be considerably alleviated.

Various attempts have been made to condition insufflation gas by heating, humidifying and or filtering the gas. However in general, known insufflation gas conditioning systems suffer from one or more disadvantages including complexity of construction involving expensive monitoring devices, inaccurate control and/or difficulties in using them in a controlled working environment.

Some systems employ heat moisture exchangers (HME). These operate directly in the flow path of the insufflation gas and are therefore inherently susceptible to affecting pressure or flow, dependent upon their level of saturation and condition. Other systems require manual intervention to respond to patients needs by the adding of moisture. Other prior art devices require the cumbersome procedure of passing gas over and through non-heated or heated liquid containers. Such devices present the major drawback of impeding pressure measurement in the insufflation cavity.

Systems using conventional jet nebulisers or nebulisation catheters exhibit one or more of the following disadvantages: impaction of larger particles, fogging in the body cavity thus reducing the surgeon's visibility, interference with insufflator settings increasing flow/pressure in the system.

This invention is directed towards providing a method and an apparatus that will address at least some of these problems.

### STATEMENTS OF INVENTION

According to the invention there is provided an apparatus for use in laparoscopic surgery comprising:
an insufflator for generating an insufflation gas;
an aerosol generator for aerosolising a fluid and entraining the aerosol with the insufflation gas wherein the aerosol generator comprises a vibratable member having a plurality of apertures extending between a first surface and a second surface; and
a controller to control the operation of the aerosol generator.

The controller may be configured to control the flow rate of the fluid to be aerosolised.

In one embodiment the controller is configured to deliver different flow rates of aerosol at different stages of a surgical procedure. The controller may be configured to deliver full flow at the start and/or end of a procedure. The controller may be configured to deliver reduced flow during a procedure.

In one embodiment the controller is set to deliver a pre-set amount of aerosol into insufflation gas. The apparatus may comprise means for varying the pre-set amount of aerosol. The means for varying the pre-set amount of aerosol may comprise a user interface such as a key pad or switch.

In one embodiment the controller is configured to control operation of the aerosol generator responsive to the insufflation gas.

The controller may be configured to control operation of the aerosol generator responsive to the flow rate of the insufflation gas.

In one case the apparatus comprises a device to determine the fluid flow rate of the insufflation gas. The determining device may comprise a flow sensor such as a flowmeter. Alternatively a differential pressure sensor may be used.

In one case a humidity meter is included in the circuit, preferably close to the patient to measure the level of humidification of the gas entering the body. In this case a feedback loop to the controller may be provided to control output from the nebulizer so as to ensure sufficient humidity is present in the insufflation gas. Such a system can be used to provide real time measurement to adjust the output from the nebulizer.

In one embodiment the first surface of the vibratable member is adapted to receive the fluid to be aerosolised.

The aerosol generator is configured to generate an aerosol at the second surface of the vibratable member.

In one embodiment the vibratable member is dome-shaped in geometry. Alternatively it may be of stretched flat shape.

In one case the vibratable member comprises a piezoelectric element.

The apertures in the vibratable member are sized to aerosolise the first fluid by ejecting droplets of the first fluid such that the majority of the droplets by mass have a size of less than 5 micrometers. The apertures in the vibratable member may be sized to aerosolise the first fluid by ejecting droplets of the first fluid such that the majority of the droplets by mass have a size of less than 3 micrometers.

The apertures in the vibratable member may be sized to aerosolise the first fluid by ejecting droplets of the first fluid such that the majority of the droplets by mass have a size in a particular predetermined range of less than 10 micrometers. In one case the range may be in a band of 1 to 3 microns and also a different range band of for example 7-9 microns.

In one case the controller is configured to control the pulse rate at a set frequency of vibration of the vibratable member.

The controller may be impedance matched to the aerosol generator.

In one embodiment the apparatus comprises means to determine whether the fluid is in contact with the aerosol generator.

The determining means may be configured to determine at least one electrical characteristic of the aerosol generator. The determining means may be configured to determine at least one electrical characteristic of the aerosol generator over a range of vibration frequencies.

In one case the determining means is configured to compare the at least one electrical characteristic against a pre-defined set of data.

The invention also provides a method for carrying out a procedure involving insufflation comprising the steps of:—
generating an insufflation gas;
aerosolising a fluid using an aerosol generator wherein the aerosol generator comprises a vibratable member having a plurality of apertures extending between a first surface and a second surface; and
entraining the aerosol with the insufflation gas.

The method may comprise the step of controlling the aerosolisation of the fluid.

The method may comprise controlling the flow rate of the fluid.

In one embodiment the method comprises delivering different flow rates of aerosol at different stages of a surgical procedure. The method may comprise delivering full flow at the start and/or end of a procedure. The method may comprise delivering reduced flow during a procedure.

In one embodiment the method comprises delivering a pre-set amount of aerosol into insufflation gas. The method may comprise the step of varying the pre-set amount. An interface may be operated to vary the pre-set amount.

In one case the method comprises controlling aerosolisation of the fluid responsive to the insufflation gas so as to ensure adequate humidification of the insufflation gas. The amount of water a gas can hold is known. Consequently, nebulizer output when linked to insufflator flow can be used to provide sufficient aerosol to humidify the gas.

In one case a humidity meter is included in the circuit, preferably close to the patient to measure the level of humidification of the gas entering the body. In this case a feedback loop to the controller may be provided to control output from the nebulizer so as to ensure sufficient humidity is present in the insufflation gas. Such a system can be used to provide real time measurement to adjust the output from the nebulizer.

In one case the method comprises controlling aerosolisation of the fluid responsive to the flow rate of the insufflation gas.

In one embodiment the method comprises the step of determining the flow rate of the insufflation gas.

In another embodiment the method comprises the step of determining if the fluid is in contact with an aerosol generator. This may involve determining at least one electrical characteristic of the aerosol generator. Electrical characteristics of the aerosol generator may be determined over a range of vibration frequencies.

In one case the method comprises the step of comparing the at least one electrical characteristic against a pre-defined set of data.

In one embodiment the method comprises the step of delivering the entrained fluid and insufflation gas into a body to insufflate at least part of the body.

In one case the fluid is an aqueous solution.

The aqueous solution may be saline having a salt concentration >1 μM

In one embodiment the fluid contains a therapeutic and/or prophylactic agent. The agent may be one or more selected from the group comprising an analgesic, an anti-inflammatory, an anti-infective, an anaesthetic, an anti-cancer chemotherapy agent, and/or anti-adhesion agent.

In one case the procedure is a laparoscopic procedure.

### BRIEF DESCRIPTION OF THE DRAWINGS

The invention will be more clearly understood from the following description of some embodiments thereof, given by way of example only, with reference to the accompanying drawings, in which:—

FIG. 1 is a perspective view of an apparatus according to the invention for use in a procedure involving insufflation of a body cavity, such as laparoscopic surgery;

FIG. 2 is a schematic illustration of a part of an apparatus according to the invention;

FIG. 3 is a schematic illustration of a part of the apparatus of FIG. 1;

FIG. 4 is an exploded isometric view of an aerosol generator used in the invention;

FIG. 5 is a cross-sectional view of the assembled aerosol generator of FIG. 4;

FIG. 6 is a perspective view of a controller housing used in the apparatus of the invention;

FIGS. 7(a) and 7(b) are graphs of DC voltage versus time and AC voltage versus time respectively to achieve a 100% aerosol output;

FIGS. 8(a) and 8(b) are graphs of DC voltage versus time and AC voltage versus time respectively to achieve a 50% aerosol output—FIG. 8(a) illustrates the waveform output from a microprocessor to a drive circuit and FIG. 8(b) illustrates the waveform output from a drive circuit to a nebuliser;

FIGS. 9(a) and 9(b) are graphs of DC voltage versus time and AC voltage versus time respectively to achieve a 25% aerosol output —FIG. 9(a) illustrates the waveform output from a microprocessor to a drive circuit and FIG. 9(b) illustrates the waveform output from a drive circuit to a nebuliser;

FIG. 10 is a graph of AC voltage versus time; and illustrates an output waveform from a drive circuit to a nebuliser;

FIG. 11 is a graph of frequency versus current for another apparatus according to the invention;

FIG. 12 is a view similar to FIG. 1 of another apparatus of the invention;

FIG. 13 is a view similar to FIG. 1 of a further apparatus of the invention;

FIG. 14 is a view similar to FIG. 1 of a still further apparatus of the invention;

FIG. 15 is a view similar to FIG. 1 of another apparatus of the invention;

FIG. 16 is a view similar to FIG. 1 of a further apparatus of the invention;

FIG. 17 is a partially cross sectional view of a detail of the apparatus of FIG. 16;

FIG. 18 is a view similar to FIG. 1 of another apparatus of the invention; and

FIG. 19 is an enlarged view of a detail of the apparatus of FIG. 18.

### DETAILED DESCRIPTION

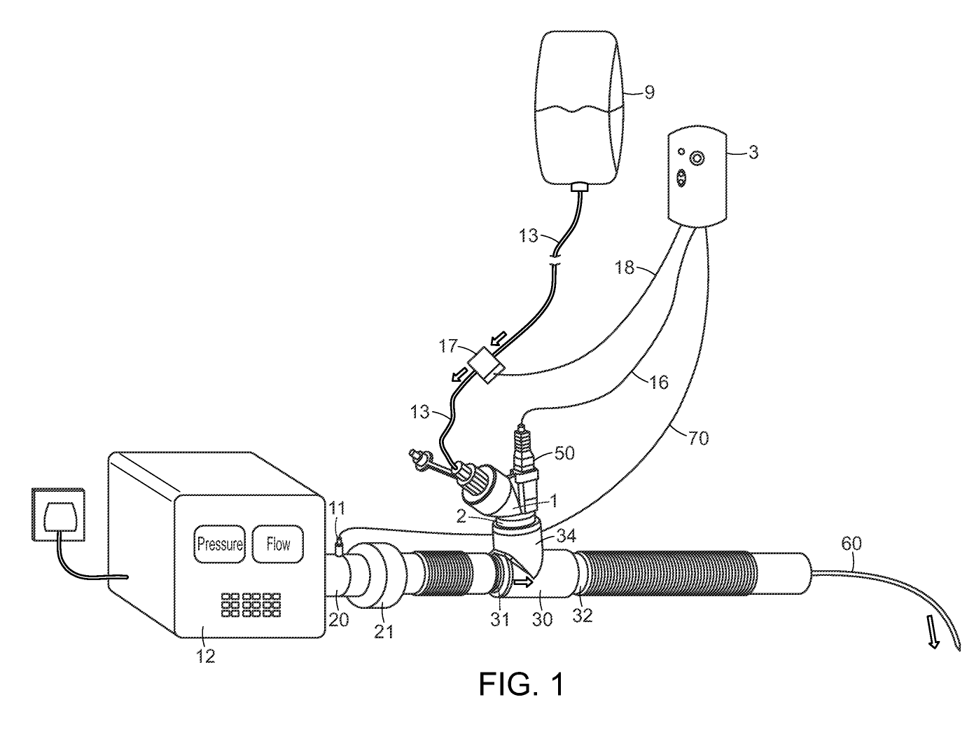

Referring to FIG. 1 there is illustrated an apparatus according to the invention for use in insufflation of a body cavity. One such application is laparoscopic surgery. The device is also suitable for use in any situation involving insufflation of a body cavity such as in arthroscopies, pleural cavity insufflation (for example during thoracoscopy), retroperitoneal insufflations (for example retroperitoneoscopy), during hernia repair, during mediastinoscopy and any other such procedure involving insufflation.

The apparatus comprises a reservoir 1 for storing an aqueous solution, an aerosol generator 2 for aerosolising the solution, and a controller 3 for controlling operation of the aerosol generator 2. The aqueous solution is fed from a reservoir 9 to the aerosol generator 2 along a delivery tube 13. In the invention aerosolised aqueous solution is entrained with insufflation gas. The gas is any suitable insufflation gas such as carbon dioxide. Other examples of suitable insufflation gases are nitrogen, helium and xenon.

The insufflation gas is delivered into an insufflation gas tubing 15 by an insufflator 12. The insufflator 12 may be of any suitable type such as those available from Karl Storz, Olympus and Stryker. The insufflator 12 has an outlet 20 through which insufflation gas is delivered. A bacterial filter 21 may be provided within the insufflator or, as illustrated, downstream of the insufflator outlet 20.

In this case a flow rate sensor/meter 11 is located in the flow path of the insufflation gas from an insufflator 12 to the aerosol generator 2. The flow rate sensor/meter 11 is connected by a control wire 70 to the controller 3, and the aerosol generator 2 is connected to the controller 3 by a control wire 16. The flow rate sensor/meter 11 may be a hot wire anemometer, or in the case where the flow is laminar or can be laminarised, a differential pressure transducer.

Sterile water may be used. In the case of an aqueous solution any suitable solution may be used. Solutions with a salt concentration in the range 1 μM (micro molar) to 154 mM (milli molar) (0.9% saline) are optimum as they cover the majority of medical applications. In addition, such saline concentrations can be readily nebulised using the aerosolisation technology used in the invention.

Liquid, saline or water for humidifying purposes only and/or medicament, can be delivered into the nebulizer reservoir through the opening in the top of the nebulizer that is appropriately sized to receive standard nebules or alternatively may be applied by syringe or other delivery means. In another embodiment it would be possible to supply the nebulizer pre-loaded with medicament avoiding the requirement to separately add medicament to the system.

Aqueous solution may be stored in the reservoir 1 container of the nebuliser or the aqueous solution may be delivered to the reservoir 1 of the aerosol generator 2 in this case from the supply reservoir 9 along the delivery line 13. The flow of aqueous solution may be by gravity and/or may be assisted by an in-line flow controlling device 17 such as a pump and/or a valve which may be positioned in the delivery line 13. The operation of the flow controlling device 17 may be controlled by the controller 3 along a control wire 18 to ensure that the aerosol generator 2 has a supply of aqueous solution during operation. The device 17 may be of any suitable type.

The apparatus comprises a connector 30, in this case a T-piece connector 30 having an insufflation gas conduit inlet 31 and an outlet 32. The connector 30 also comprises an aerosol supply conduit 34 for delivering the aerosol from the aerosol generator 2 into the insufflation gas conduit 15 to entrain the aerosol with the insufflation gas, passing through the gas insufflation conduit 15. The entrained aerosol/insufflation gas mixture passes out of the connector 30 through the outlet 32 and is delivered to the body cavity along a line 60.

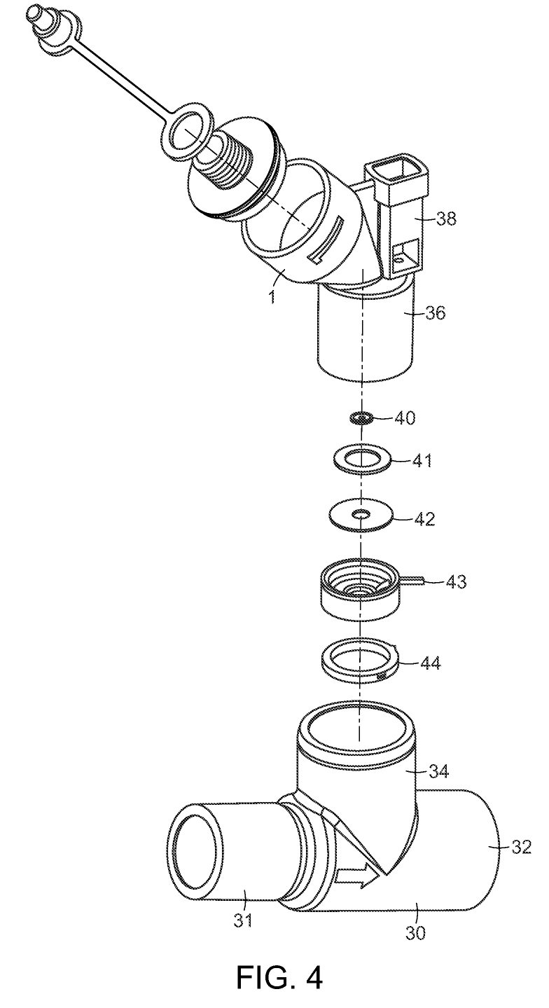

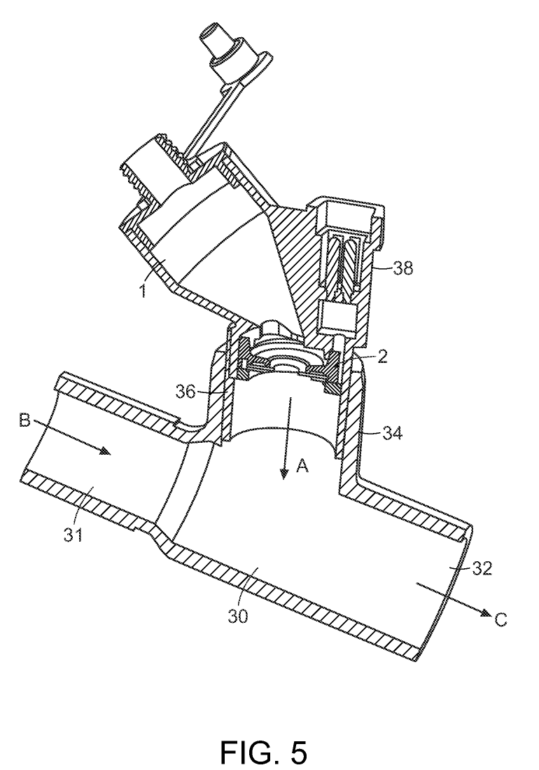

The aerosol supply conduit 34 and the insufflation gas conduit meet at a junction. Referring particularly to FIGS. 4 and 5, in the assembled apparatus the aerosol supply conduit of the connector 30 may be releasably mounted to a neck 36 of the aerosol generator housing by means of a push-fit arrangement. This enables the connector 30 to be easily dismounted from the aerosol generator housing 36, for example for cleaning. The neck 36 at least partially lines the interior of the aerosol supply conduit 34.

The nebuliser (or aerosol generator), has a vibratable member which is vibrated at ultrasonic frequencies to produce liquid droplets. Some specific, non-limiting examples of technologies for producing fine liquid droplets is by supplying liquid to an aperture plate having a plurality of tapered apertures extending between a first surface and a second surface thereof and vibrating the aperture plate to eject liquid droplets through the apertures. Such technologies are described generally in U.S. Pat. Nos. 5,164,740; 5,938,117; 5,586,550; 5,758,637; 6,014,970, 6,085,740, and US2005/021766A, the complete disclosures of which are incorporated herein by reference. However, it should be appreciated that the present invention is not limited for use only with such devices.

Various methods of controlling the operation of such nebulisers or aerosol generators are described in U.S. Pat. No. 6,540,154, U.S. Pat. No. 6,845,770, U.S. Pat. No. 5,938,117 and U.S. Pat. No. 6,546,927, the complete disclosures of which are incorporated herein by reference.

In use, the liquid to be aerosolised is received at the first surface, and the aerosol generator 2 generates the aerosolised first fluid at the second surface by ejecting droplets of the first fluid upon vibration of the vibratable member. The apertures in the vibratable member are sized to aerosolise the liquid by ejecting droplets of the liquid such that the majority of the droplets by mass have a size of less than 5 micrometers. The vibratable member 40 could be non-planar, and may be dome-shaped in geometry.

Referring particularly to FIGS. 4 and 5, in one case the aerosol generator 2 comprises a vibratable member 40, a piezoelectric element 41 and a washer 42, which are sealed within a silicone overmould 43 and secured in place within the housing 36 using a retaining ring 44. The vibratable member 40 has a plurality of tapered apertures extending between a first surface and a second surface thereof.

The first surface of the vibratable member 40, which in use faces upwardly, receives the liquid medicament from the reservoir 1 and the aerosolised medicament, is generated at the second surface of the vibratable member 40 by ejecting droplets of medicament upon vibration of the member 40. In use the second surface faces downwardly. In one case, the apertures in the vibratable member 40 may be sized to produce an aerosol in which the majority of the droplets by weight have a size of less than 5 micrometers.

The complete nebuliser may be supplied in sterile form, which is a significant advantage for a surgical device.

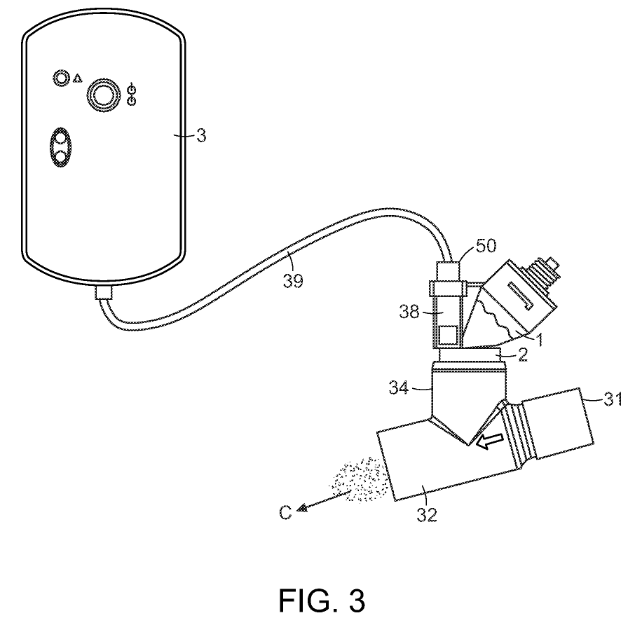

Referring particularly to FIG. 3, the controller 3 controls operation of and provides a power supply to the aerosol generator 2. The aerosol generator has a housing which defines the reservoir 1. The housing has a signal interface port 38 fixed to the lower portion of the reservoir 1 to receive a control signal from the controller 3. The controller 3 may be connected to the signal interface port 38 by means of a control lead 39 which has a docking member 50 for mating with the port 38. A control signal and power may be passed from the controller 3 through the lead 39 and the port 38 to the aerosol generator 2 to control the operation of the aerosol generator 2 and to supply power to the aerosol generator 2 respectively.

The power source for the controller 3 may be an on-board power source, such as a rechargeable battery, or a remote power source, such as a mains power source, or an insufflator power source. When the remote power source is an AC mains power source, an AC-DC converter may be connected between the AC power source and the controller 3. A power connection lead may be provided to connect a power socket of the controller 3 with the remote power source.

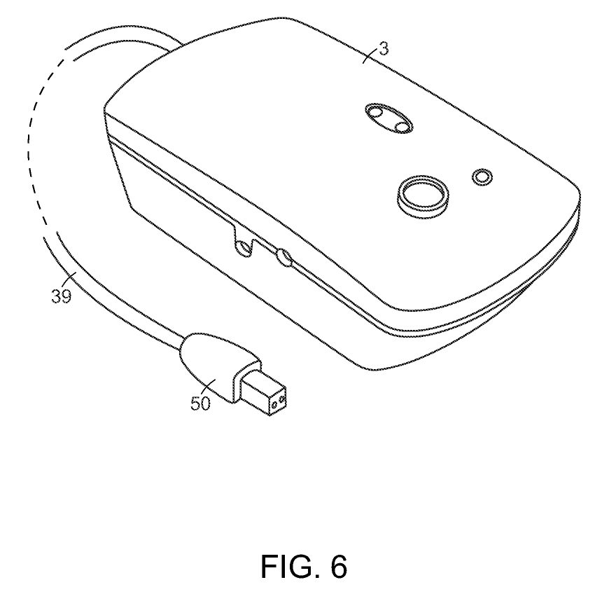

Referring particularly to FIG. 6 the controller 3 has a housing and a user interface to selectively control operation of the aerosol generator 2. Preferably the user interface is provided on the housing which, in use, is located remote from the aerosol generator housing. The user interface may be in the form of, for example, an on-off button. In one embodiment a button can be used to select pre-set values for simplicity of use. In another embodiment a dial mechanism can be used to select from a range of values from 0-100%.

Status indication means are also provided on the housing to indicate the operational state of the aerosol generator 2. For example, the status indication means may be in the form of two visible LED's, with one LED being used to indicate power and the other LED being used to indicate aerosol delivery. Alternatively one LED may be used to indicate an operational state of the aerosol generator 2, and the other LED may be used to indicate a rest state of the aerosol generator 2.

A fault indicator may also be provided in the form of an LED on the housing. A battery charge indicator in the form of an LED may be provided at the side of the housing.

Referring particularly to FIG. 1, the aqueous solution in the reservoir 9 flows by gravitational action towards the aerosol generator 2 at the lower medicament outlet. The controller 3 may then be activated to supply power and a control signal to the aerosol generator 2, which causes the piezoelectric element 41 to vibrate the non-planar member 40. This vibration of the non-planar member 40, causes the aqueous solution at the top surface of the member 40 to pass through the apertures to the lower surface where the aqueous solution is aerosolised by the ejection of small droplets of solution.

Referring particularly to FIGS. 4 and 5, the aerosol passes from the aerosol generator 2 into the neck 36 of the aerosol generator housing, which is mounted within the aerosol supply conduit of the connector 30 and into the gas conduit of the connector 30 (flow A). The aerosol is entrained in the insufflation gas conduit with gas, which passes into the gas conduit through the inlet 31 (flow B). The entrained mixture of the aerosol and the insufflation gas then passes out of the gas conduit through the outlet 32 (flow C) and on via an insufflator line 60 to a patient, for example into the abdomen of the patient.

In use during laparoscopic surgery the flow of the insufflation gas into the abdomen of a patient is commenced to insufflate the abdomen. The flow rate sensor/meter 11 determines the flow rate of the insufflation gas. In response to the fluid flow rate of the insufflation gas, the controller 3 commences operation of the aerosol generator 2 to aerosolise the aqueous solution. The aerosolised aqueous solution is entrained with the insufflation gas, and delivered into the abdomen of the patient to insufflate at least part of the abdomen.

In the event of alteration of the fluid flow rate of the insufflation gas, the flow rate sensor/meter 11 determines the alteration, and the controller 3 alters the pulse rate of the vibratable member of the nebuliser accordingly.

The controller 3 is in communication with the flow rate sensor/meter 11. The controller 3 is configured to control operation of the aerosol generator 2, responsive to the fluid flow rate of the insufflation gas and also independent of the fluid flow rate of the insufflation gas as required.

In one case, the controller 3 is configured to control operation of the aerosol generator 2 by controlling the pulse rate at a set frequency of vibration of the vibratable member, and thus controlling the fluid flow rate of the aqueous solutions.

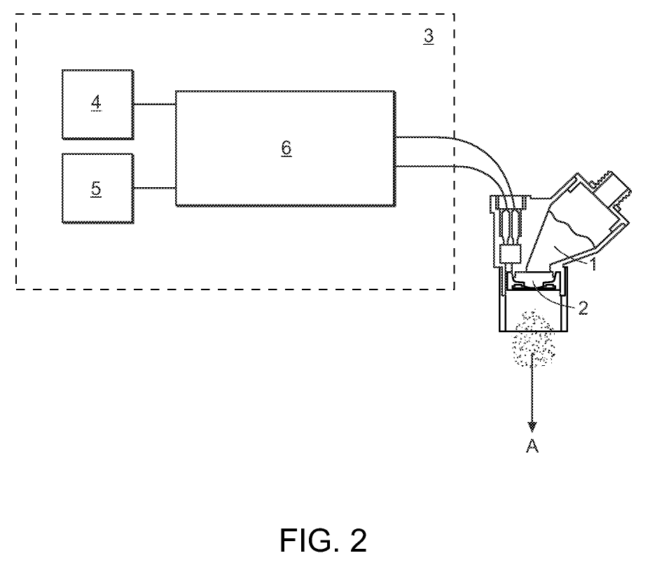

The controller 3 may comprise a microprocessor 4, a boost circuit 5, and a drive circuit 6. FIG. 2 illustrates the microprocessor 4, the boost circuit 5, the drive circuit 6 comprising impedance matching components (inductor), the nebuliser 2, and the aerosol. The inductor impedance is matched to the impedance of the piezoelectric element of the aerosol generator 2. The microprocessor 4 generates a square waveform of 128 KHz which is sent to the drive circuit 6. The boost circuit 5 generates a 12V DC voltage required by the drive circuit 6 from an input of either a 4.5V battery or a 9V AC/DC adapter. The circuit is matched to the impedance of the piezo ceramic element to ensure enhanced energy transfer. A drive frequency of 128 KHz is generated to drive the nebuliser at close to its resonant frequency so that enough amplitude is generated to break off droplets and produce the aerosol. If this frequency is chopped at a lower frequency such that aerosol is generated for a short time and then stopped for a short time this gives good control of the nebuliser's flow rate. This lower frequency is called the pulse rate.

The drive frequency may be started and stopped as required using the microprocessor 4. This allows for control of flow rate by driving the nebuliser 2 for any required pulse rate. The microprocessor 4 may control the on and off times to an accuracy of milliseconds.

The nebuliser 2 may be calibrated at a certain pulse rate by measuring how long it takes to deliver a know quantity of solution. There is a linear relationship between the pulse rate and the nebuliser flow rate. This allows for accurate control over the delivery rate of the aqueous solution.

The nebuliser drive circuit consists of the electronic components designed to generate output sine waveform of approximately 100V AC which is fed to nebuliser 2 causing aerosol to be generated. The nebuliser drive circuit 6 uses inputs from microprocessor 4 and boost circuit 5 to achieve its output. The circuit is matched to the impedance of the piezo ceramic element to ensure good energy transfer.

The aerosol generator 2 may be configured to operate in a variety of different modes, such as continuous, and/or phasic, and/or optimised.

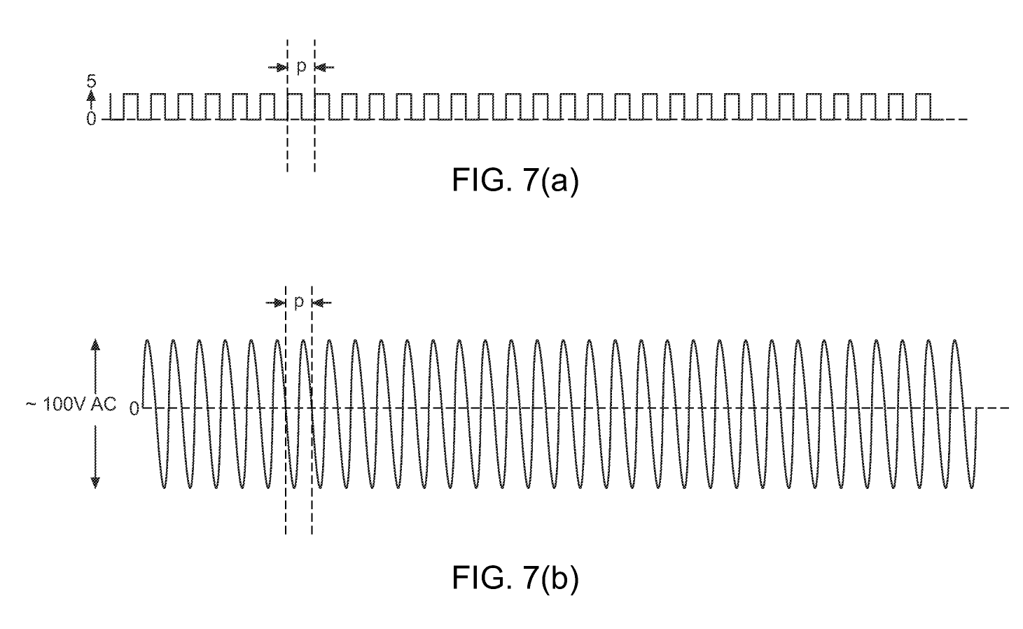

For example, referring to FIG. 7(a) illustrates a 5V DC square waveform output from the microprocessor 4 to the drive circuit 6. FIG. 7(b) shows a low power, ˜100V AC sine waveform output from drive circuit 6 to nebuliser 2. Both waveforms have a period p of 7.8 μS giving them a frequency of 1/7.8 μs which is approximately 128 KHz. Both waveforms are continuous without any pulsing. The aerosol generator may be operated in this mode to achieve 100% aerosol output.

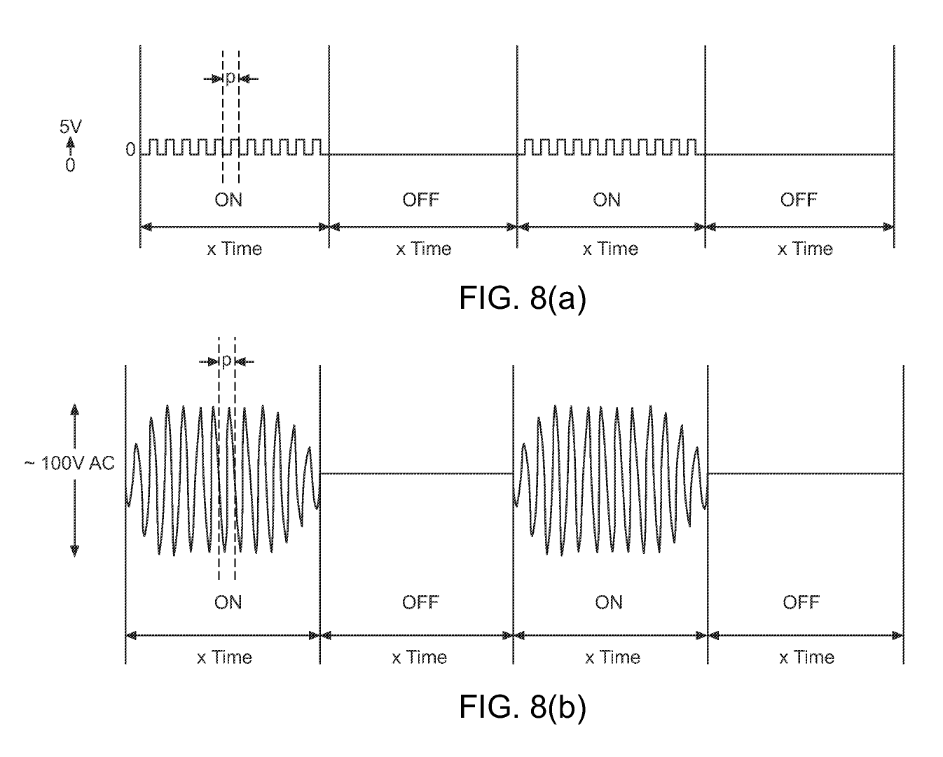

Referring to FIG. 8(a) in another example, there is illustrated a 5V DC square waveform output from the microprocessor 4 to the drive circuit 6. FIG. 8(b) shows a low power, ˜100V AC sine waveform output from the drive circuit 6 to the nebuliser 2. Both waveforms have a period p of 7.8 μS giving them a frequency of 1/7.8 μs which is approximately 128 KHz. In both cases the waveforms are chopped (stopped/OFF) for a period of time x. In this case the off time x is equal to the on time x. The aerosol generator may be operated in this mode to achieve 50% aerosol output.

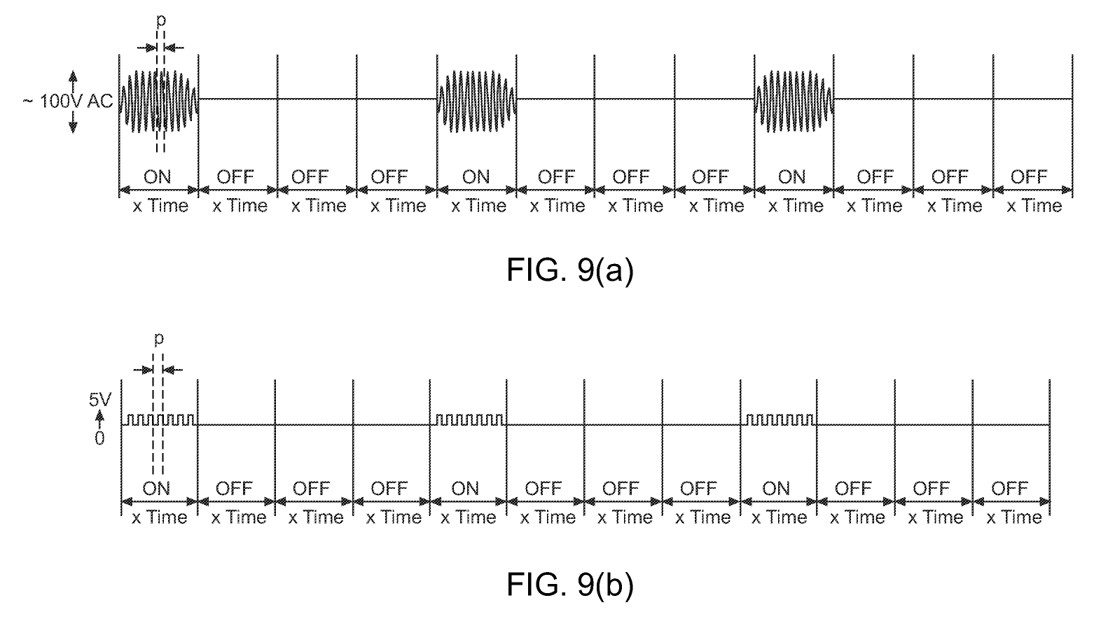

In another case, referring to FIG. 9(a) there is illustrated a 5V DC square waveform output from microprocessor 4 to drive circuit 6. FIG. 9(b) shows a low power, ˜100V AC sine waveform output from the drive circuit 6 to the nebuliser 2. Both waveforms have a period p of 7.8 μS giving them a frequency of 1/7.8 μs which is approximately 128 KHz. In both cases the waveforms are chopped (stopped/OFF) for a period of time x. In this case the off time is 3× while the on time is x. The aerosol generator may be operated in this mode to achieve 25% aerosol output.

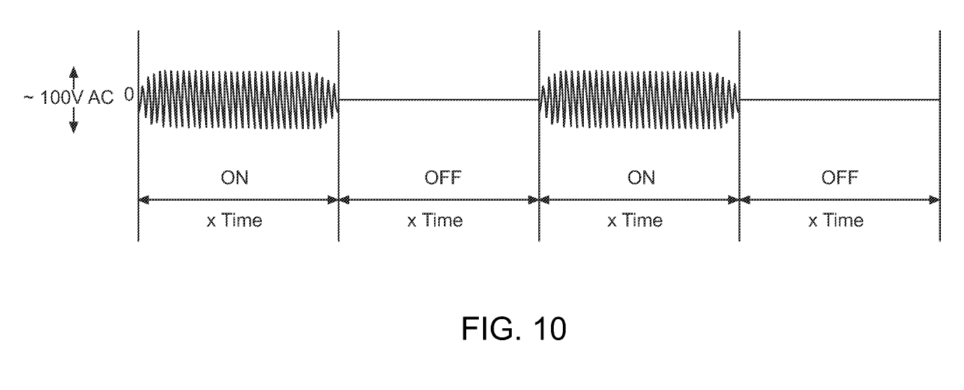

Referring to FIG. 10, in one application pulsing is achieved by specifying an on-time and off-time for the vibration of the aperture plate. If the on-time is set to 200 vibrations and off-time is set to 200 vibrations, the pulse rate is 50% (½ on ½ off). This means that the flow rate is half of that of a fully driven aperture plate. Any number of vibrations can be specified but to achieve a linear relationship between flow rate and pulse rate a minimum number of on-time vibrations is specified since it takes a finite amount of time for the aperture plate to reach its maximum amplitude of vibrations.

The drive frequency can be started and stopped as required by the microprocessor; this allows control of flow rate by driving the nebuliser for any required pulse rate. The microprocessor can control the on and off times with an accuracy of microseconds.

A nebuliser can be calibrated at a certain pulse rate by measuring how long it takes to deliver a known quantity of solution. There is a linear relationship between the pulse rate and that nebuliser's flow rate. This allows accurate control of the rate of delivery of the aerosolised aqueous solution. The ability to calibrate each nebulizer ensures that any inherent variation in output rate between each nebulizer can be eliminated. The output from each nebulizer when in-line in the insufflator circuit will be equivalent to a second nebulizer although the inherent flow rates of the two nebulizers are different. For example, to achieve a standard output of 0.044 ml/min at 1 Lmin from two nebulizers, one with an inherent output of 0.088 ml/min and a second with an inherent output of 0.176 ml/min the first nebulizer is controlled with a 50:50 on:off pulse rate, with the second set to a 25:75 on-off pulse rate so that both nebulizers give a 0.044 ml/min output. This feature ensures that the nebulizers when located in the insufflation circuit have the potential to provide exactly the same rate of aerosol output as each other. This is possible because the amount of humidity a gas can hold is a known constant dependent on controllable factors.

The pulse rate may be lowered so that the velocity of the emerging aerosol is much reduced so that impaction rain-out is reduced.

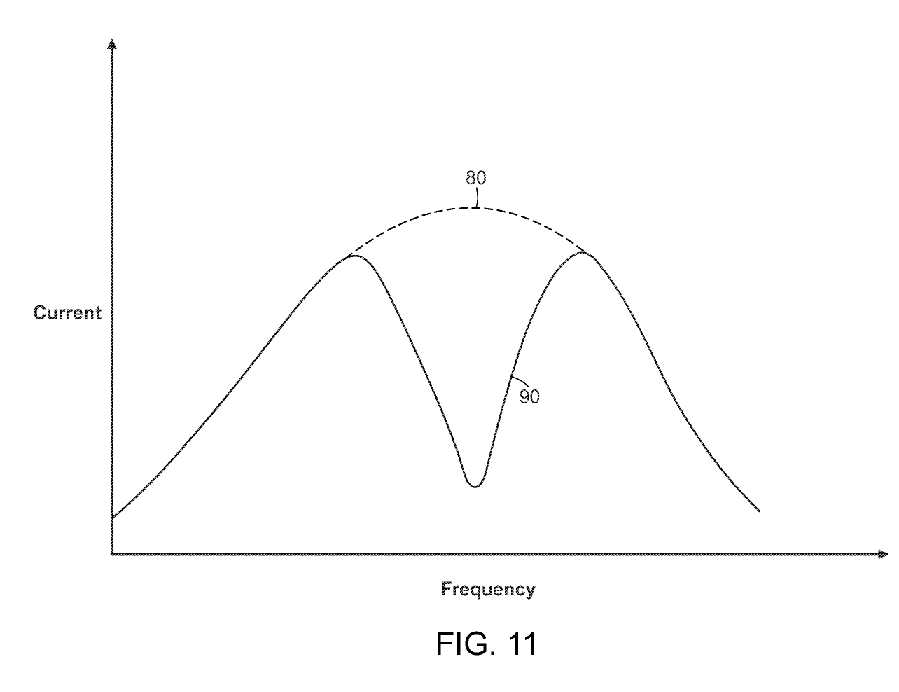

Detection of when the aperture plate is dry can be achieved by using the fact that a dry aperture plate has a well defined resonant frequency. If the drive frequency is swept from 120 kHz to 145 kHz and the current is measured then if a minimum current is detected less than a set value, the aperture plate must have gone dry. A wet aperture plate has no resonant frequency. The apparatus of the invention may be configured to determine whether there is any of the first fluid in contact with the aerosol generator 2. By determining an electrical characteristic of the aerosol generator 2, for example the current flowing through the aerosol generator 2, over a range of vibration frequencies, and comparing this electrical characteristic against a pre-defined set of data, it is possible to determine whether the aerosol generator 2 has any solution in contact with the aerosol generator 2. FIG. 11 illustrates a curve 80 of frequency versus current when there is some of the solution in contact with the aerosol generator 2, and illustrates a curve 90 of frequency versus current when there is none of the solution in contact with the aerosol generator 2. FIG. 11 illustrates the wet aperture plate curve 80 and the dry aperture plate curve 90.

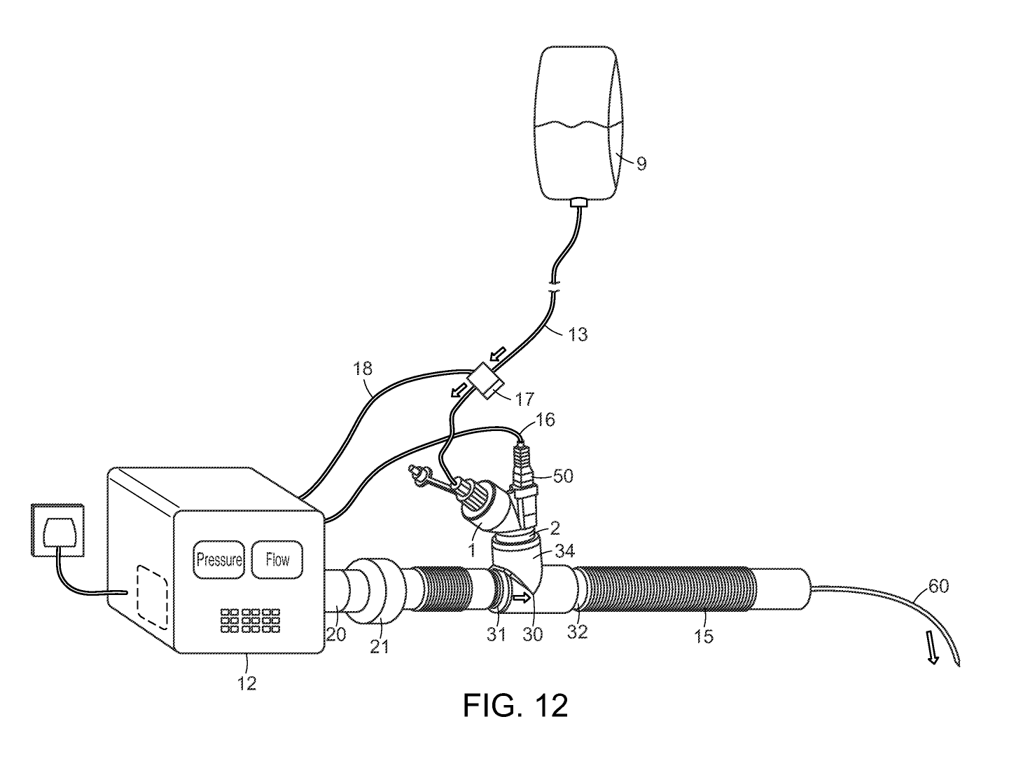

If an application requires a constant feed from a drip bag then a pump can be added in line to give fine control of the liquid delivery rate which can be nebulised drip by drip. The rate would be set so that liquid would not build up in the nebuliser. This system is particularly suitable for constant low dose delivery. Referring now to FIG. 12 there is illustrated another insufflation apparatus which is similar to the apparatus of FIG. 1 and like parts are arranged the same reference numerals. In this case the controller 3 is integrated into the insufflator 12. The insufflator 12 would have information on the rate of flow that it is producing and using an integrated circuit board may directly communicate with the nebuliser 2. This would eliminate the need for the separate flowmeter 11 and the stand-alone controller 3 to be present.

In another case there may be a common information bus between the insufflator 12 and the controller 3. The insufflator 12 would have information on the rate of flow that it is producing and would communicate this to the controller 3 and on to the nebuliser 2, thereby eliminating the need for the flowmeter 11. This would allow the invention to be backward compatible with a variety of types of insufflator.

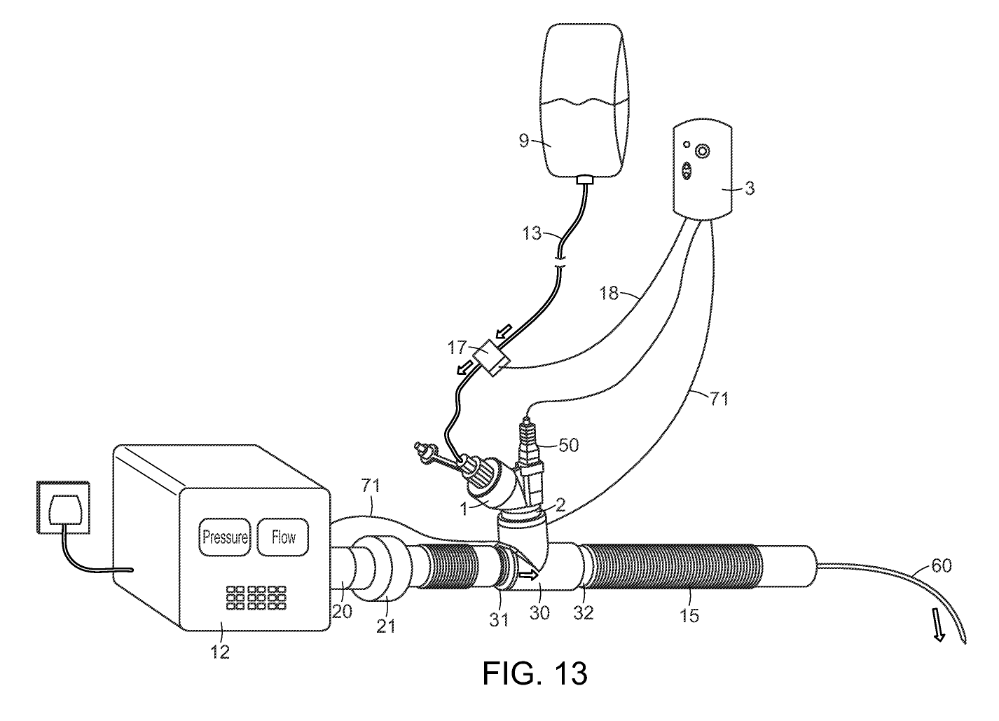

Referring to FIG. 13 there is illustrated another insufflation apparatus which is similar to the apparatus of FIG. 1 and like parts are again identified by the same reference numerals. In this case the insufflation gas flow signal is provided directly from the insufflator along a lead 71. One advantage of this arrangement is that no separate meter/sensor required.

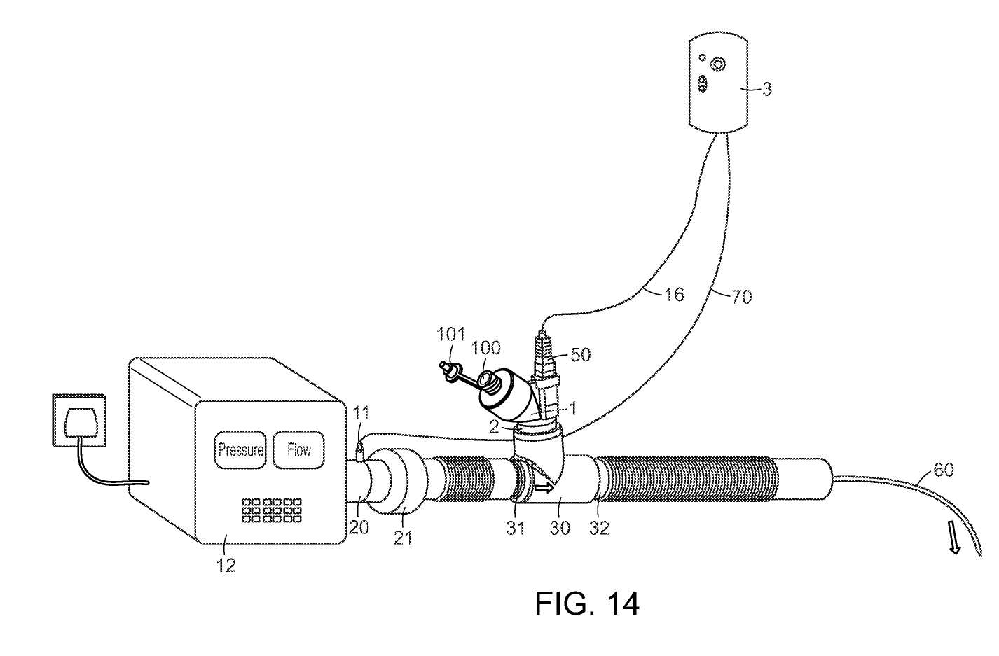

Referring to FIG. 14 there is illustrated another apparatus according to the invention which is similar to that illustrated in FIG. 1 and like parts are assigned the same reference numerals. In this case the nebuliser reservoir 1 has a top opening 100 which is closable by removable plug 101. Liquid, saline or water for humidifying purposes and/or medicament is delivered into the nebuliser reservoir through the opening 100. The opening 100 is appropriately sized to receive standard nebules containing liquid to be nebulised. The liquid may be applied by syringe or other suitable delivery means.

It is also possible to provide the nebuliser 1 pre-loaded with medicament to avoid the requirement to separately add medicament to the system.

The apparatus of FIG. 14 is operated in a similar way to the modes of operation described above with reference to FIGS. 2 to 11.

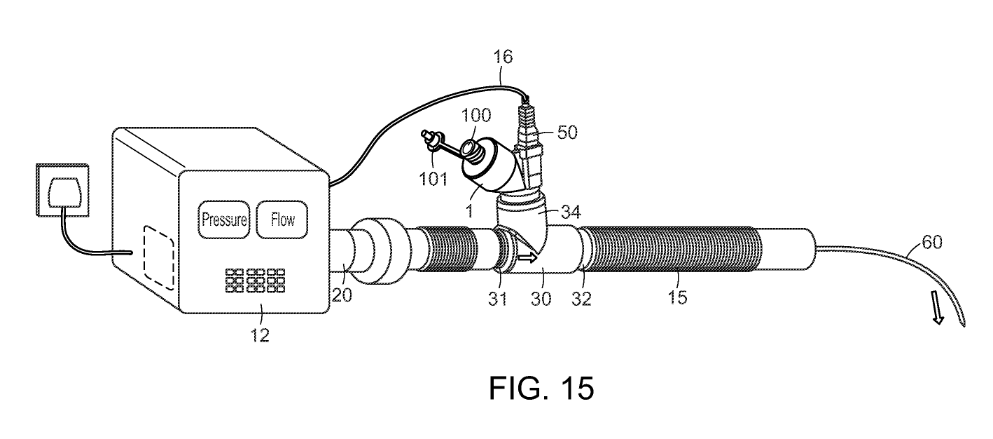

Referring to FIG. 15 there is illustrated another apparatus of the invention which is similar to that described above with reference to FIG. 12 and like parts are assigned the same reference numerals. In this case the nebuliser reservoir 1 has a top opening 100 and a removable plug/lid 101 as described with reference to FIG. 14 and the apparatus is operated as described above with the liquid being introduced through the opening 100. Again the nebuliser may be pre-loaded with medicament.

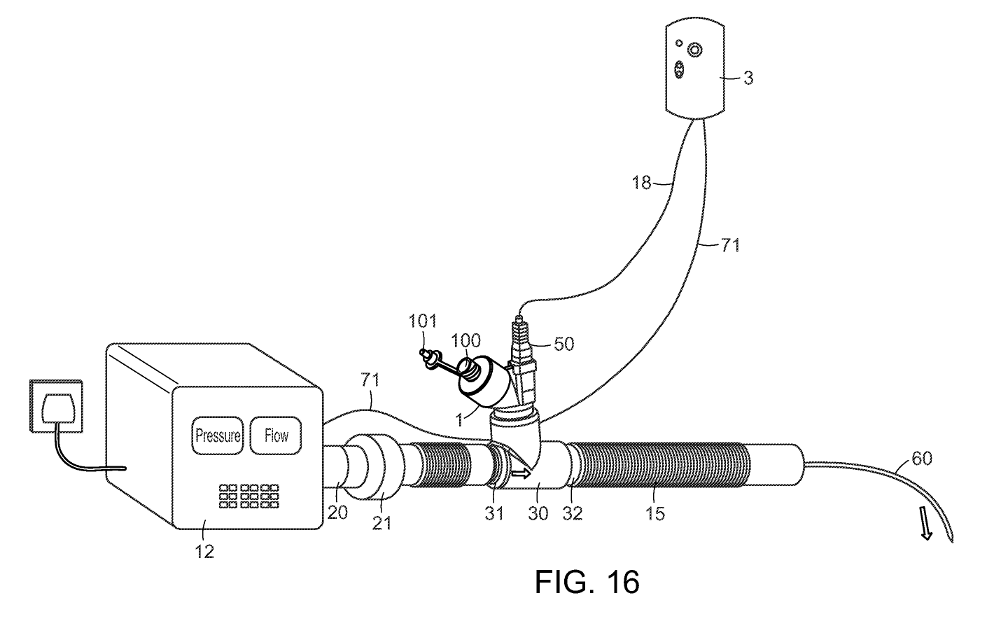

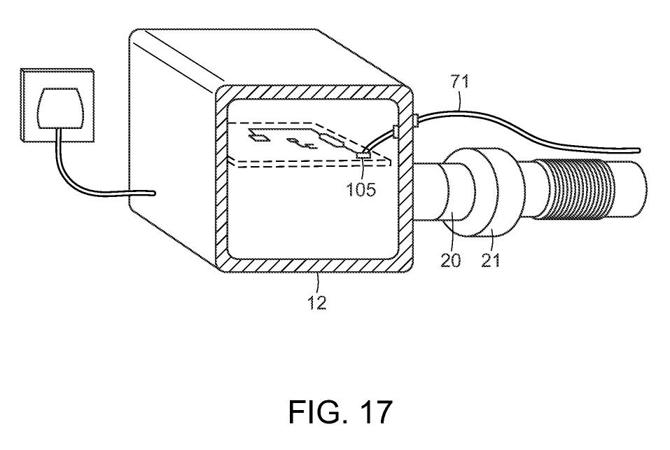

Referring to FIG. 16 there is illustrated another apparatus of the invention which is similar to that described above with reference to FIG. 13 and like parts are assigned the same reference numerals. In this case the nebuliser reservoir 1 again has a top opening 100 and a removable lid 101 as described with reference to FIG. 14 and the apparatus is operated as described above with the liquid being introduced through the opening 100. The nebuliser may be pre-loaded with medicament. The apparatus is operated as described above. FIG. 17 shows the connection of the controller lead 71 to the control circuit 105 of the insufflator 12.

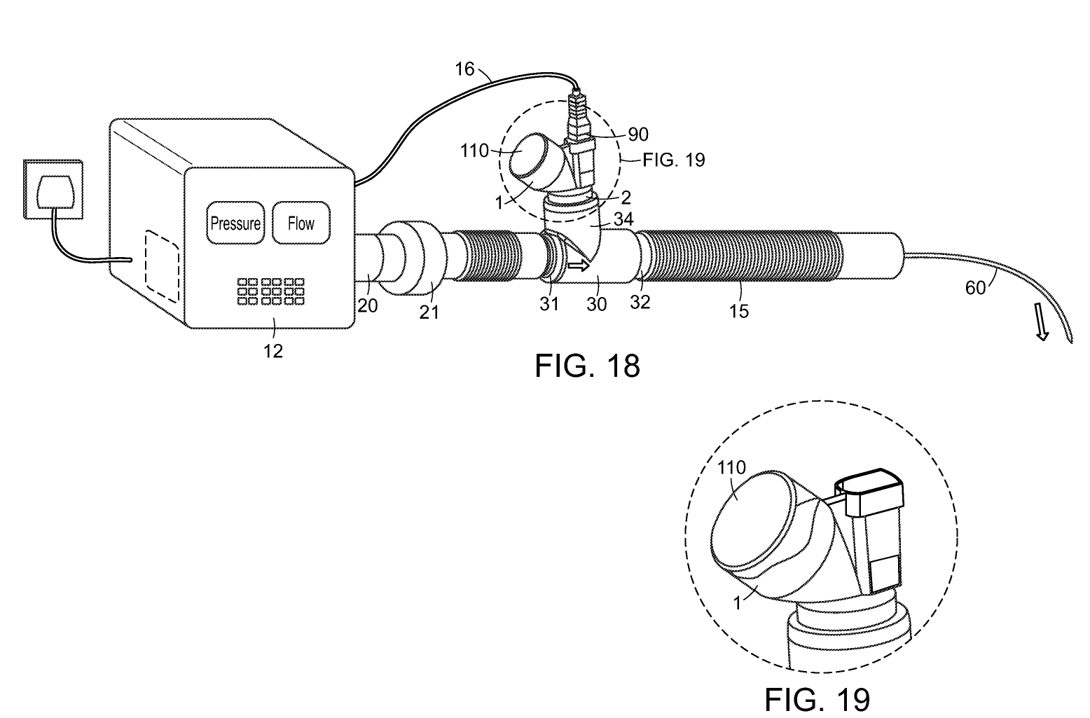

Referring to FIGS. 18 and 19 there is illustrated a further apparatus according to the invention which is similar to those described above and like parts are assigned the same reference numerals. In this case the nebuliser reservoir 1 is closed by a lid 110 and the nebuliser is pre-loaded with medicament/liquid which avoids the requirement to separately add medicament to the system.

Humidity may be generated via the aerosolisation of any aqueous solution. Relative humidity in the 50-100% range would be optimum. The control module can generate a nebuliser output of any defined relative humidity percentage based on the insufflator flow. These solutions include any aqueous drug solution. Solutions with salt concentrations in the range 1 μM-154 mM would be optimum.

The use of the nebulizer to humidify the insufflation gas prior to entering the body will eliminate the need for the body to humidify the gas once it is inside the body, thereby minimizing body heat loss by internal evaporation.

The control in nebulizer output allows proportional delivery of the required amount of humidity according to the amount of insufflation gas entering the body. In addition this control of aerosolization rate will prevent overloading of the insufflation gas with aerosol which would obscure the surgeons view.

The invention provides a system that can deliver different flow rates at different stages of the surgical procedure. Examples of such different flow rates include:
(i) delivering at 100% at the start of the procedure (Bolus);
(ii) delivering at a much lower rate say 5% during the procedure itself (Lower flow rate avoid fogging);
(iii) delivering at 100% at the end of the procedure (Bolus);
(iv) any combination of the above sequencing with variable % values.

In one case the controller which controls the operation of the aerosol generator is pre-set to deliver a set amount of aerosol into the insufflation gas. For example, the controller may be set to deliver an amount of 5% into a flow of 1 litre per minute of insufflation gas to avoid fogging. The controller may be pre-set in the factory to operate in this manner. Alternatively there may be a user interface such as a switch, or keypad which may be used to change the setting. In these arrangements control responsive to an insufflation gas flow sensor is not essential.

In addition to acting as a humidifying agent the nebulizer can also act to deliver any agent presented in an aqueous drug solution. The system facilitates delivery of, for example, pain-relief medications, anti-infectives, anti-inflammatory and/or chemotherapy agents in aerosol form to the body cavity. These therapeutic agents could also act as humidifying substances in their own right.

The nebulised liquid entrained in the insufflation gas may contain any desired therapeutic and/or prophylactic agent. Such an agent may for example be one or more of an analgesic, an anti-inflammatory, an anaesthetic, an anti-infective such as an antibiotic, an anti-cancer chemotherapy agent, and/or any agent which interferes with processes that result in the adhesion function.

Typical local anaesthetics are, for example, Ropivacaine, Bupivacaine and Lidocaine.

Typical anti-infectives include antibiotics such as an aminoglycoside, a tetracycline, a fluoroquinolone; anti-microbials such as a cephalosporin; and anti-fungals.

Anti-inflammatories may be of the steroidal or non-steroidal type.

Anti-cancer chemotherapy agents may be alkylating agents, antimetabolites anthracyclines, plant alkaloids, topoisomerase inhibitors, nitrosoureas, mitotic inhibitors, monoclonal antibodies, tyrosine kinase inhibitors, hormone therapies including corticosteroids, cancer vaccines, anti-estrogens, aromatase inhibitors, anti-androgens, anti-angiogenic agents and other antitumour agents.

The agent which interferes with the adhesion function may be any of those outlined in WO2005/092264A, the entire contents of which are herein incorporated by reference. In particular, the agent may be a crystalloid, hyaluronic acid, polyethyleneglycol, Tranilast (N-(3¹,4¹-dimethoxycinnamoyl) anthranilic acid) or a Neurokinin 1 receptor (NK-1R) agonist, such as Aprepitant.

Typical analgesics include aspirin, acetaminophen, ibuprofen, naproxen, a Cox-2 inhibitor such as celecoxib, morphine, oxycodone and hydrocodone.

The system of the invention can be used for precise controlled delivery of drug and/or humidity during insufflation. No heating is required. Consequently there is no risk of damage to drugs due to heating The system may be used to provide precise control over aerosol output can be exercised by utilising pulse rate control.

The system may be used for targeted delivery of a range of drugs, thereby reducing systemic side effects. In addition the system provides alleviation of post-surgical pain experienced by the patient.

The system need not be located in the direct flow path of insufflation gas. In addition, minimal caregiver intervention during laparoscopic procedure is required. The system is small and compact and allows for integration with an insufflator.

The device of the invention can be used throughout the procedure carried out by a surgeon. The device ensures that humidity is actively controlled during the procedure and thus ensures that a surgeon's view is clear as fogging is avoided.

In the system of the invention the nebuliser output is controlled by pulsing to provide delivery of humidity and/or medicament into the insufflation gas during surgery without causing fogging.

The control may be provided either by providing a maximum output limit on the nebuliser or by linking directly to the insufflator flow.

All parts of the device (except the controller and associated leads) are autoclavable which provides a significant advantage for a device used in surgery.

The invention is not limited to the embodiments hereinbefore described which may be varied in construction and detail.

1. Apparatus for use in insufflation comprising:
an insufflator for generating an insufflation gas;
an aerosol generator for aerosolising a fluid and entraining the aerosol with the insufflation gas wherein the aerosol generator comprises a vibratable member having a plurality of apertures extending between a first surface and a second surface; and
a controller to control the operation of the aerosol generator.

2. An apparatus as claimed in claim 1 wherein the controller is configured to control the flow rate of the fluid to be aerosolised.

3. Apparatus as claimed in claim 2 wherein the controller is configured to deliver different flow rates of aerosol at different stages of a surgical procedure.

4. Apparatus as claimed in claim 3 wherein the controller is configured to deliver full flow at the start and/or end of a procedure.

5. Apparatus as claimed in claim 3 wherein the controller is configured to deliver reduced flow during a procedure.

6. Apparatus as claimed in claim 1 wherein the controller is set to deliver a pre-set amount of aerosol into insufflation gas.

7. Apparatus as claimed in claim 6 comprising means for varying the pre-set amount of aerosol.

8. Apparatus as claimed in claim 7 wherein the means for varying the pre-set amount of aerosol comprises a user interface such as a keypad or switch.

9. An apparatus as claimed in claim 1 wherein the controller is configured to control operation of the aerosol generator responsive to the insufflation gas.

10. An apparatus as claimed in claim 9 wherein the controller is configured to control operation of the aerosol generator responsive to the flow rate of the insufflation gas.

11. An apparatus as claimed in claim 9 wherein the apparatus comprises a device to determine the fluid flow rate of the insufflation gas.

12. An apparatus as claimed in claim 11 wherein the determining device comprises a flow sensor.

13. An apparatus as claimed in claim 12 wherein the flow sensor comprises a flowmeter.

14. An apparatus as claimed in claim 11 wherein the device to determine the fluid flow rate comprises a differential pressure sensor.

15. An apparatus as claimed in claim 9 comprising a humidity meter to measure the level of humidification of the insufflation gas.

16. An apparatus as claimed in claim 15 comprising a feedback loop to the controller to control the output from the aerosol generator responsive to the level of humidification of the insufflation gas.

17. An apparatus as claimed in claim 1 wherein the first surface is adapted to receive the fluid to be aerosolised.

18. An apparatus as claimed in claim 1 wherein the aerosol generator is configured to generate an aerosol at the second surface.

19. An apparatus as claimed in claim 1 wherein the vibratable member is dome-shaped in geometry.

20. An apparatus as claimed in claim 1 wherein the vibratable member comprises a stretched flat shape.

21. An apparatus as claimed in claim 1 wherein the vibratable member comprises a piezoelectric element.

22. An apparatus as claimed in claim 1 wherein the apertures in the vibratable member are sized to aerosolise the first fluid by ejecting droplets of the first fluid such that the majority of the droplets by mass have a size of less than 5 micrometers.

23. An apparatus as claimed in claim 1 wherein the apertures in the vibratable member are sized to aerosolise the first fluid by ejecting droplets of the first fluid such that the majority of the droplets by mass have a size of less than 3 micrometers.

24. An apparatus as claimed in claim 1 wherein the apertures in the vibratable member are sized to aerosolise the first fluid by ejecting droplets of the first fluid such that the majority of the droplets by mass have a size in one range of less than 10 micrometers.

25. An apparatus as claimed in claim 24 wherein a range band is from 1 to 3 micrometers.

26. An apparatus as claimed in claim 24 wherein a range band is from 7 to 9 micrometers.

27. An apparatus as claimed in claim 1 wherein the controller is configured to control the pulse rate at a set frequency of vibration of the vibratable member.

28. An apparatus as claimed in claim 1 wherein the controller is impedance matched to the aerosol generator.

29. An apparatus as claimed in claim 1 wherein the apparatus comprises means to determine whether the fluid is in contact with the aerosol generator.

30. An apparatus as claimed in claim 29 wherein the determining means is configured to determine at least one electrical characteristic of the aerosol generator.

31. An apparatus as claimed in claim 30 wherein the determining means is configured to determine at least one electrical characteristic of the aerosol generator over a range of vibration frequencies.

32. An apparatus as claimed in claim 30 wherein the determining means is configured to compare the at least one electrical characteristic against a pre-defined set of data.

33. A method for carrying out a procedure involving insufflation comprising the steps of:—
generating an insufflation gas;
aerosolising a fluid using an aerosol generator wherein the aerosol generator comprises a vibratable member having a plurality of apertures extending between a first surface and a second surface; and
entraining the aerosol with the insufflation gas.

34. A method as claimed in claim 33 comprising controlling the flow rate of the fluid.

35. A method as claimed in claim 34 comprising the step of controlling the aerosoliation of the fluid.

36. A method as claimed in claim 35 comprising delivering different flow rates of aerosol at different stages of a surgical procedure.

37. A method as claimed in claim 36 comprising delivering full flow at the start and/or end of a procedure.

38. A method as claimed in claim 36 comprising delivering reduced flow during a procedure.

39. A method as claimed in claim 34 comprising delivering a pre-set amount of aerosol into insufflation gas.

40. A method as claimed in claim 39 comprising the step of varying the pre-set amount.

41. A method as claimed in claim 40 comprising operating an interface to vary the pre-set amount.

42. A method as claimed in claim 33 comprising controlling aerosolisation of the fluid responsive to the insufflation gas.

43. A method as claimed in claim 42 comprising controlling aerosolisation of the fluid responsive to the flow rate of the insufflation gas.

44. A method as claimed in claim 42 wherein the method comprises the step of determining the flow rate of the insufflation gas.

45. A method as claimed in claim 42 comprising controlling the aerosolisation of the fluid responsive to the level of humidification of the insufflation gas.

46. A method as claimed in claim 42 wherein the method comprises the step of determining if the fluid is in contact with an aerosol generator.

47. A method as claimed in claim 46 comprising determining at least one electrical characteristic of the aerosol generator.

48. A method as claimed in claim 47 comprising determining at least electrical characteristics of the aerosol generator over a range of vibration frequencies.

49. A method as claimed in claim 47 wherein the method comprises the step of comparing the at least one electrical characteristic against a pre-defined set of data.

50. A method as claimed in claim 33 wherein the method comprises the step of delivering the entrained fluid and insufflation gas into the body to insufflate at least part of the body.

51. A method as claimed in claim 33 wherein the fluid is an aqueous solution.

52. A method as claimed in claim 51 wherein the aqueous solution is saline having a salt concentration of greater than 1 μM.

53. A method as claimed in claim 33 wherein the fluid contains a therapeutic and/or prophylactic agent.

54. A method as claimed in claim 53 wherein the agent is one or more selected from the group comprising an analgestic, an anti-inflammatory, an anti-infective, an anaesthetic, an anticancer chemotherapy agent, and an anti-adhesion agent.

55. A method as claimed in claim 33 wherein the procedure is a laparascopic procedure.
